(19) United States
(12) Patent Application Publication
(10) Pub. No.: US 2023/0090389 A1
(43) Pub. Date: Mar. 23, 2023

(54) A TRIPLE PHARMACEUTICAL COMBINATION COMPRISING DABRAFENIB, AN ERK INHIBITOR AND A SHP2 INHIBITOR

(71) Applicant: Novartis AG, Basel (CH)

(72) Inventor: Diana GRAUS PORTA, Basel, MA (US)

(21) Appl. No.: 17/905,032

(22) PCT Filed: Feb. 26, 2021
(86) PCT No.: PCT/IB2021/051643

§ 371 Date: Aug. 25, 2022

Related U.S. Application Data
(60) Provisional application No. 62/983,021, filed on Feb. 28, 2020.

(51) Int. Cl. A61K 31/506; A61K 31/497; A61K 31/4965

(52) U.S. Cl. CPC A61K 31/506; A61K 31/497; A61K 31/4965

(57) ABSTRACT

The present invention relates to a pharmaceutical combination comprising dabrafenib, an Erk-inhibitor and a SHP2 inhibitor; pharmaceutical compositions comprising the same; and methods of using such combinations and compositions in the treatment or prevention of conditions in which MAPK pathway inhibition is beneficial, for example, in the treatment of cancers.

### FIELD OF THE INVENTION

The present invention relates to a pharmaceutical combination comprising dabrafenib, or a pharmaceutically acceptable salt thereof, an Erk inhibitor (ERKi) such as 4-(3-amino-6-((1S,3S,4S)-3-fluoro-4-hydroxycyclohexyl)pyrazin-2-yl)-N-((S)-1-(3-bromo-5-fluorophenyl)-2-(methylamino)ethyl)-2-fluorobenzamide (“Compound A” or “compound A”), or a pharmaceutically acceptable salt thereof, and a SHP2 inhibitor (SHP2i) such as (3 S,4S)-8-(6-amino-5-((2-amino-3-chloropyridin-4-yl)thio)pyrazin-2-yl)-3-methyl-2-oxa-8-azaspiro[4.5]decan-4-amine (“Compound B”) or a pharmaceutically acceptable salt thereof; pharmaceutical compositions comprising the same; commercial packages comprising the same; and methods of using such combinations and compositions in the treatment or prevention of conditions in which MAPK pathway inhibition is beneficial, for example, in the treatment of cancers. The present invention also povides such combinations for use in the treatments of such conditions or cancers, including colorectal cancer (CRC) such as BRAF gain of function colorectal cancer.

### BACKGROUND OF THE INVENTION

The MAPK pathway is a key signaling cascade that drives cell proliferation, differentiation, and survival. Dysregulation of this pathway underlies many instances of tumorigenesis. Aberrant signaling or inappropriate activation of the MAPK pathway has been shown in multiple tumor types and can occur through several distinct mechanisms, including activating mutations in RAS and BRAF. The MAPK pathway is frequently mutated in human cancer with KRAS and BRAF mutations being among the most frequent (approximately 30%). RAS mutations, particularly gain of function mutations, have been detected in 9-30% of all cancers, with KRAS mutations having the highest prevalence (86%).

The extracellular signal-regulated kinases (ERKs) are one class of signaling kinases that are involved in conveying extracellular signals into cells and subcellular organelles. ERK1 and ERK2 are involved in regulating a wide range of activities and dysregulation of the ERK1/2 cascade is known to cause a variety of pathologies including neurodegenerative diseases, developmental diseases, diabetes and cancer. The role of ERK1/2 in cancer is of special interest because activating mutations upstream of ERK1/2 in its signaling cascade are believed to be responsible for more than half of all cancers. Moreover, excessive ERK1/2 activity was also found in cancers where the upstream components were not mutated, suggesting that ERK1/2 signaling plays a role in carcinogenesis even in cancers without mutational activations. The ERK pathway has also been shown to control tumor cell migration and invasion, and thus may be associated with metastasis.

The prognosis for patients suffering from certain cancers remains poor. Resistance to treatment occurs frequently and not all patients respond to available treatments. For example, the median survival for patients suffering from advanced colorectal cancer with BRAF mutation is less than 12 months. It is thus important to develop new therapies for patients suffering from cancer to achieve better clinical outcomes. Treatment options which are better tolerated and/or provide durable anti-tumor responses are also desired.

### SUMMARY OF THE INVENTION

The triple combination of the present invention: dabrafenib; an Erk-inhibitor such as Compound A; and a SHP2-inhibitor such as compound B; can be used as therapies for the treatment of diseases or disorders resulting from the aberrant activity of the MAPK pathway including, but not limited to, breast cancer, cholangiocarcinoma, salivary gland cancer, colorectal cancer, melanoma, non-small cell lung cancer, ovarian cancer and thyroid cancer. Triple combinations of dabrafenib, an Erk-inhibitor such as Compound A, and a SHP2-inhibitor such as compound B, are particularly useful in the treatment of colorectal cancer (CRC), including advanced or metastatic colorectal cancer, which is BRAF gain of function or BRAFV600E mutant.

The present invention provides a pharmaceutical combination comprising:

(a) N-(3-(5-(2-aminopyrimidin-4-yl)-2-(tert-butyl)thiazol-4-yl)-2-fluorophenyl)-2,6-difluorobenzenesulfonamide (dabrafenib), or a pharmaceutically acceptable salt thereof, having the structure:

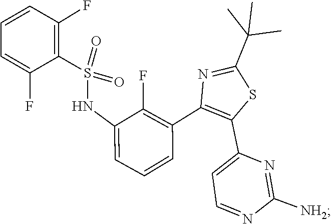 [SMILES: CC(C)(C)c1nc(-c2cccc(NS(=O)(=O)c3c(F)cccc3F)c2F)c(-c2ccnc(N)n2)s1]

(b) 4-(3-amino-6-((1S,3 S,4S)-3-fluoro-4-hydroxycyclohexyl)pyrazin-2-yl)-N-((S)-1-(3-bromo-5-fluorophenyl)-2-(methylamino)ethyl)-2-fluorobenzamide (Compound A), or a pharmaceutically acceptable salt thereof, having the structure:

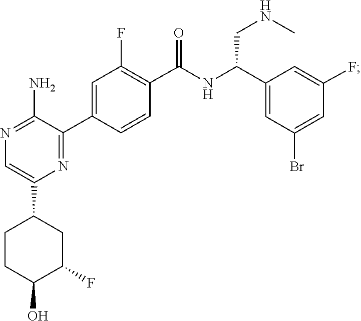 [SMILES: CNC[C@@H](NC(=O)c1ccc(-c2nc([C@H]3CC[C@H](O)[C@@H](F)C3)cnc2N)cc1F)c1cc(F)cc(Br)c1]

and (c) (3S,4S)-8-(6-amino-5-((2-amino-3-chloropyridin-4-yl)thio)pyrazin-2-yl)-3-methyl-2-oxa-8-azaspiro[4.5]decan-4-amine (Compound B), or a pharmaceutically acceptable salt thereof, having the structure:

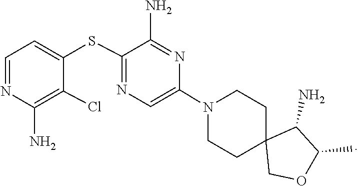 [SMILES: C[C@@H]1OCC2(CCN(c3cnc(Sc4ccnc(N)c4Cl)c(N)n3)CC2)[C@@H]1N]

Pharmaceutical combinations of dabrafenib, or a pharmaceutically acceptable salt thereof, Compound A, or a pharmaceutically acceptable salt thereof, and Compound B, or a pharmaceutically acceptable salt thereof, will also be referred to herein as a “combination of the invention”.

There is provided a combination of the invention for use in the treatment of cancer, e.g for use in a cancer which is selected from breast cancer, cholangiocarcinoma, salivary gland cancer, colorectal cancer, melanoma, non-small cell lung cancer, ovarian cancer and thyroid cancer.

There is provided a pharmaceutical combination of dabrafenib, or a pharmaceutically acceptable salt thereof, Compound A, or a pharmaceutically acceptable salt thereof, and Compound B, or a pharmaceutically acceptable salt thereof, e.g for use in a cancer which is selected from breast cancer, cholangiocarcinoma, salivary gland cancer, colorectal cancer, melanoma, non-small cell lung cancer, ovarian cancer and thyroid cancer. There is also provided a combination of combination of dabrafenib, or a pharmaceutically acceptable salt thereof, Compound A, or a pharmaceutically acceptable salt thereof, and Compound B, or a pharmaceutically acceptable salt thereof, for use in the treatment of colorectal cancer (which includes advanced or metastsatic colorectal cancer) which is BRAF gain of function or BRAFV600E mutant.

Also provided herein is a combination of the invention for use in the treatment of colorectal cancer (which includes advanced or metastsatic colorectal cancer) which is BRAF gain of function or BRAFV600E mutant.

In another embodiment of the combination of the invention, dabrafenib, or a pharmaceutically acceptable salt thereof, Compound A, or a pharmaceutically acceptable salt thereof, and Compound B, or a pharmaceutically acceptable salt thereof, and are in the same formulation.

In another embodiment of the combination of the invention, dabrafenib, or a pharmaceutically acceptable salt thereof, Compound A, or a pharmaceutically acceptable salt thereof, and Compound B, or a pharmaceutically acceptable salt thereof, are in separate formulations.

In another embodiment, the combination of the invention is for simultaneous or sequential (in any order) administration.

In another embodiment, the present invention provides a method for treating cancer in a subject in need thereof comprising administering to the subject a therapeutically effective amount of the combination of the invention.

In a further embodiment of the method, the cancer is selected from breast cancer, cholangiocarcinoma, salivary gland cancer, colorectal cancer, melanoma, non-small cell lung cancer, ovarian cancer and thyroid cancer.

In a further embodiment, the present invention provides a combination of the invention for use in the manufacture of a medicament for treating a cancer selected from breast cancer, cholangiocarcinoma, salivary gland cancer, colorectal cancer, melanoma, non- small cell lung cancer, ovarian cancer and thyroid cancer.

In another embodiment there is provided a pharmaceutical composition or commercial package (e.g. a kit-of-parts) comprising the combination of the invention.

In a further embodiment, the pharmaceutical composition further comprises one or more pharmaceutically acceptable excipients.

### BRIEF DESCRIPTION OF THE DRAWINGS

FIG. 1: Combination activity of MAPK pathway inhibitors in BRAF-mutant CRC cell lines. Six BRAF-mutated CRC cell lines were treated with the either Compound B alone, dabrafenib+compound A doublet or dabrefenib+Compound A+Compound B triplet. The graph shows the percentage of growth inhibition (% GI) achieved after seven treatment days with respect to DMSO-treated cells. The % GI values are average values of independent experiments and the vertical error bars indicate the standard deviation. The horizontal dotted line indicates 100% GI (cell stasis). Values extending beyond 100% GI indicate cell kill.

### DESCRIPTION

The general terms used hereinbefore and hereinafter preferably have within the context of this disclosure the following meanings, unless otherwise indicated, where more general terms whereever used may, independently of each other, be replaced by more specific definitions or remain, thus defining more detailed embodiments of the invention:

“Dabrafenib” is N-(3-(5-(2-aminopyrimidin-4-yl)-2-(tert-butyl)thiazol-4-yl) fluorophenyl)-2,6-difluorobenzenesulfonamide, a selective inhibitor of mutated BRAF at V600 capable of inhibiting BRAF(V600E), BRAF(V600K) and BRAF(V600G) mutations, (also known as: N-{3-[5-(2-Amino-4-pyrimidinyl)-2-(1,1-dimethylethyl)-1,3-thiazol-4-yl]-2-fluorophenyl}-2,6-difluorobenzenesulfonamide; Tafinlar®; & N-{3[5-(2-Amino-4-pyrimidinyl)-2-(1,1-dimethyle thyl)-1,3-thiazol-4-yl]-2-fluorophenyl}-2,6 difluorobenzene sulfonamide, methanesulfonate salt).

“Cetuximab” is an epidermal growth factor receptor (EGFR) inhibitor used for the treatment of metastatic colorectal cancer, metastatic non-small cell lung cancer and head and neck cancer. Cetuximab is an epidermal growth factor receptor-targetedIgG1 monoclonal antibody that is approved for use in combination with irinotecan or as monotherapy in the treatment of metastatic CRC. Cetuximab is a chimeric (mouse/human) monoclonal antibody given by intravenous infusion.

“Compound A” is an inhibitor of extracellular signal-regulated kinases (ERK) 1/2. “Compound A” is 4-(3-amino-6-((1S,3S,4S)-3-fluoro-4-hydroxycyclohexyl)pyrazin-2-yl)-N-((S)-1-(3-bromo-5-fluorophenyl)-2-(methylamino)ethyl)-2-fluorobenzamide. A particularly preferred salt of Compound A is the hydrochloride salt thereof.

“Compound B” is an inhibitor of SHP2. Compound B is (3S,4S)-8-(6-amino-5-((2-amino-3-chloropyridin-4-yl)thio)pyrazin-2-yl)-3-methyl-2-oxa-8-azaspiro[4.5]decan-4-amine. A particularly preferred salt of Compound B is the succinate salt.

SHP2 inhibitors include compound B (above) and compounds described in WO2015/107493, WO2015/107494, WO2015/107495, WO2016/203406, WO2016/203404, WO2016/203405, WO2017/216708, WO2018/013597, WO2018/136264, WO2018/13265, WO2019/051084, WO2019/075265, WO2019/118909, WO2019/199792, WO2017/211303, WO2018/172984, WO2017/156397, WO2018/057884, WO2018/081091, WO2019/067843, WO2019/165073 & WO2019/183367.

The term “subject” or “patient” as used herein is intended to include animals, which are capable of suffering from or afflicted with a cancer or any disorder involving, directly or indirectly, a cancer. Examples of subjects include mammals, e.g., humans, apes, monkeys, dogs, cows, horses, pigs, sheep, goats, cats, mice, rabbits, rats, and transgenic non-human animals. In an embodiment, the subject is a human, e.g., a human suffering from, at risk of suffering from, or potentially capable of suffering from cancers.

The term “treating” or “treatment” as used herein comprises a treatment relieving, reducing or alleviating at least one symptom in a subject or effecting a delay of progression of a disease. For example, treatment can be the diminishment of one or several symptoms of a disorder or complete eradication of a disorder, such as cancer. Within the meaning of the present disclosure, the term “treat” also denotes to arrest, delay the onset (i.e., the period prior to clinical manifestation of a disease) and/or reduce the risk of developing or worsening a disease.

The terms “comprising” and “including” are used herein in their open-ended and non-limiting sense unless otherwise noted.

The terms “a” and “an” and “the” and similar references in the context of describing the invention (especially in the context of the following claims) are to be construed to cover both the singular and the plural, unless otherwise indicated herein or clearly contradicted by context. Where the plural form is used for compounds, salts, and the like, this is taken to mean also a single compound, salt, or the like.

By “a combination” or “in combination with” or “co-administration with” and such like, it is not intended to imply that the therapy or the therapeutic agents must be physically mixed or administered at the same time and/or formulated for delivery together, although these methods of delivery are within the scope described herein. A therapeutic agent in these combinations can be administered concurrently with, prior to, or subsequent to, one or more other additional therapies or therapeutic agents. The therapeutic agents or therapeutic protocol can be administered in any order. In general, each agent will be administered at a dose and/or on a time schedule determined for that agent. It will further be appreciated that the additional therapeutic agent utilized in this combination may be administered together in a single composition or administered separately in different compositions. In general, it is expected that additional therapeutic agents utilized in combination be utilized at levels that do not exceed the levels at which they are utilized individually. In some embodiments, the levels utilized in combination will be lower than those utilized as single-agent therapeutics.

When describing a dosage or dose herein as ‘about’ a specified amount, the actual dosage or dose can vary by up to 10%, e.g. 5%, from the stated amount: this usage of ‘about’ recognizes that the precise amount in a given dose or dosage form may differ slightly from an intended amount for various reasons without materially affecting the in vivo effect of the administered compound. The skilled person will understand that where a dose or dosage of a therapeutic compound is quoted herein, that amount refers to the amount of the therapeutic compound in its free form or unsolvated form.

The phrase “therapeutically-effective amount” as used herein means that amount of a compound, material, or composition comprising a compound of the present invention which is effective for producing some desired therapeutic effect in at least a sub-population of cells in an animal (including a human) at a reasonable benefit/risk ratio applicable to any medical treatment.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The combinations of the invention, dabrafenib, compound A and compound B, is also intended to represent unlabeled forms as well as isotopically labeled forms of the compounds. Isotopically labeled compounds have one or more atoms replaced by an atom having a selected atomic mass or mass number. Examples of isotopes that can be incorporated into dabrafenib, compound A and Compound B include isotopes of hydrogen, carbon, nitrogen, oxygen, phosphorous, fluorine, and chlorine, such as ²H, ³H, ¹¹C, ¹³C, ¹⁴C, 15_{N,} ¹⁸F ³¹P, ³²P, ³⁵S, ³⁶Cl, ¹²³I, ¹²⁴I, ¹²⁵I respectively . The invention includes isotopically labeled dabrafenib, compound A and compound B, for example into which radioactive isotopes, such as ³H and ¹⁴C, or non-radioactive isotopes, such as ²H and ¹³C, are present. Isotopically labelled dabrafenib, compound A and compound B are useful in metabolic studies (with ¹⁴C), reaction kinetic studies (with, for example ²H or ³H), detection or imaging techniques, such as positron emission tomography (PET) or single-photon emission computed tomography (SPECT) including drug or substrate tissue distribution assays, or in radioactive treatment of patients. In particular, dabrafenib, compound A or compound B labeled with ¹⁸F may be particularly desirable for PET or SPECT studies. Isotopically-labeled compounds of the invention can generally be prepared by conventional techniques known to those skilled in the art or by processes analogous to those described in the accompanying Examples using appropriate isotopically-labeled reagents.

Further, substitution with heavier isotopes, particularly deuterium (i.e., ²H or D) may afford certain therapeutic advantages resulting from greater metabolic stability, for example increased in vivo half-life or reduced dosage requirements or an improvement in therapeutic index. It is understood that deuterium in this context is regarded as a substituent of either dabrafenib, compound A or compound B. The concentration of such a heavier isotope, specifically deuterium, may be defined by the isotopic enrichment factor. The term “isotopic enrichment factor” as used herein means the ratio between the isotopic abundance and the natural abundance of a specified isotope. If a substituent dabrafenib, compound A or compound B is denoted deuterium, such compound has an isotopic enrichment factor for each designated deuterium atom of at least 3500 (52.5% deuterium incorporation at each designated deuterium atom), at least 4000 (60% deuterium incorporation), at least 4500 (67.5% deuterium incorporation), at least 5000 (75% deuterium incorporation), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation).

Description of Preferred Embodiments

Dabrafenib is an orally bioavailable small molecule with RAF inhibitory activity. Compound A is an orally bioavailable small molecule with ERK inhibitory activity. It is an inhibitor of extracellular signal-regulated kinases 1 and 2 (ERK 1/2). Compound B is an orally bioavailable small molecule with SHP2 inhibitory activity.

In one embodiment, with respect to the pharmaceutical combination of the invention, is a pharmaceutical combination comprising: N-(3-(5-(2-aminopyrimidin-4-yl)-2-(tert-butyl)thiazol-4-yl)-2-fluorophenyl)-2,6-difluorobenzenesulfonamide (dabrafenib), or a pharmaceutically acceptable salt thereof; 4-(3-amino-6-((1S,3S,4S)-3-fluoro-4-hydroxycyclohexyl)pyrazin-2-yl)-N-((S)-1-(3-bromo-5-fluorophenyl)-2-(methylamino)ethyl)-2-fluorobenzamide (compound A), or a pharmaceutically acceptable salt thereof; and (3S,4S)-8-(6-amino-5-((2-amino-3-chloropyridin-4-yl)thio)pyrazin-2-yl)-3-methyl-2-oxa-8-azaspiro[4.5]decan-4-amine (compound B), or a pharmaceutically acceptable salt thereof.

In a further embodiment, N-(3-(5-(2-aminopyrimidin-4-yl)-2-(tert-butyl)thiazol-4-yl)-2-fluorophenyl)-2,6-difluorobenzenesulfonamide (dabrafenib), or a pharmaceutically acceptable salt thereof, 4-(3-amino-6-((1S,3S,4S)-3-fluoro-4-hydroxycyclohexyl)pyrazin-2-yl)-N-((S)-1-(3-bromo-5-fluorophenyl)-2-(methylamino)ethyl)-2-fluorobenzamide (compound A), or a pharmaceutically acceptable salt thereof, and (3 S,4S)-8-(6-amino-5-((2-amino-3-chloropyridin-4-yl)thio)pyrazin-2-yl)-3-methyl-2-oxa-8-azaspiro[4.5]decan-4-amine (compound B), or a pharmaceutically acceptable salt thereof, are administered separately, simultaneously or sequentially, in any order.

In a further embodiment, the pharmaceutical combination is for oral administration.

In a further embodiment of the pharmaceutical combination, N-(3-(5-(2-aminopyrimidin-4-yl)-2-(tert-butyl)thiazol-4-yl)-2-fluorophenyl)-2,6-difluorobenzenesulfonamide (dabrafenib) is in an oral dosage form.

In a further embodiment of the pharmaceutical combination, 4-(3-amino-6-((1S,3S,4S)-3-fluoro-4-hydroxycyclohexyl)pyrazin-2-yl)-N-((S)-1-(3-bromo-5-fluorophenyl)-2-(methylamino)ethyl)-2-fluorobenzamide (compound A) is in an oral dosage form.

In a further embodiment of the pharmaceutical combination, (3S,4S)-8-(6-amino-5-((2-amino-3-chloropyridin-4-yl)thio)pyrazin-2-yl)-3-methyl-2-oxa-8-azaspiro[4.5]decan-4-amine (compound B) is in an oral dosage form.

In another embodiment is a pharmaceutical composition or a commercial package comprising the pharmaceutical combination (as described in any of the embodiments above) and at least one pharmaceutically acceptable carrier.

In another embodiment is a pharmaceutical combination (as described in any of the embodiments above) or the pharmaceutical composition or the commercial package (as described in the embodiments above) for use in the treatment of cancer.

In a further embodiment, the cancer is selected from breast cancer, cholangiocarcinoma, colorectal cancer (CRC), melanoma, non-small cell lung cancer, ovarian cancer and thyroid cancer.

In a further embodiment, the cancer is advanced or metastatic colorectal cancer.

In a further embodiment, the cancer is BRAF gain of function CRC or BRAF V600E, V600D or V600K CRC.

In another embodiment is a use of the pharmaceutical combination according to any of the above embodiments or the pharmaceutical composition or commercial package according to the above embodiments for the manufacture of a medicament for the treatment of cancer.

In a further embodiment, the cancer is selected from breast cancer, cholangiocarcinoma, colorectal cancer, melanoma, non-small cell lung cancer, ovarian cancer and thyroid cancer, optionally wherein the cancer is advanced or metastatic colorectal cancer, optionally wherein the cancer is BRAF gain of function CRC or BRAF V600E, V600D or V600K CRC.

In another embodiment is a method of treating a cancer selected from breast cancer, cholangiocarcinoma, colorectal cancer, melanoma, non-small cell lung cancer, ovarian cancer and thyroid cancer comprising administrating to a patient in need thereof a pharmaceutical combination or commercial package according to any one of the above embodiemnts or the pharmaceutical composition according to the above embodiments.

In a further embodiment, the colorectal cancer is advanced or metastatic colorectal cancer.

In a further embodiment, the colorectal cancer is BRAF gain of function CRC or BRAF V600E, V600D or V600K CRC.

In a further embodiment, N-(3-(5-(2-aminopyrimidin-4-yl)-2-(tert-butyl)thiazol-4-yl)-2-fluorophenyl)-2,6-difluorobenzenesulfonamide (dabrafenib) is administered orally at a dose of about from about 1 to about 150 mg per day (for example, 1, 2, 5, 10, 50, 100 or 150 mg per day).

In a further embodiment, 4-(3-amino-6-((1S,3S,4S)-3-fluoro-4-hydroxycyclohexyl)pyrazin-2-yl)-N-((S)-1-(3-bromo-5-fluorophenyl)-2-(methylamino)ethyl)-2-fluorobenzamide (compound A) is administered orally at a dose of from about 50 to about 200 mg per day (for example, at a dose of about 50, 75, 100, 125, 150, 175 or 200 mg per day).

In a further embodiment, (3S,4S)-8-(6-amino-5-((2-amino-3-chloropyridin-4-yl)thio)pyrazin-2-yl)-3-methyl-2-oxa-8-azaspiro[4.5]decan-4-amine (compound B) is adminstered orally at a dose of from about 1.5 mg per day, or 3 mg per day, or 6 mg per day, or 10 mg per day, or 20 mg per day, or 30 mg per day, or 40 mg per day, or 50 mg per day, or 60 mg per day to about 70 mg per day.

In a further embodiment, (3S,4S)-8-(6-amino-5-((2-amino-3-chloropyridin-4-yl)thio)pyrazin-2-yl)-3-methyl-2-oxa-8-azaspiro[4.5]decan-4-amine (compound B) is adminstered orally wherein the dose per day is on a 21 day cycle of 2 weeks on drug followed by 1 week off drug.

In a further embodiment, (3S,4S)-8-(6-amino-5-((2-amino-3-chloropyridin-4-yl)thio)pyrazin-2-yl)-3-methyl-2-oxa-8-azaspiro[4.5]decan-4-amine (compound B) is adminstered orally wherein the dose per day is on a 14 day cycle of 2 weeks on drug followed by 1 week off drug.

### Pharmacology and Utility

The RAS/RAF/MEK/ERK or mitogen activated protein kinase (MAPK) pathway is a key signaling cascade that integrates upstream cellular signals, such as from growth factor receptor tyrosine kinases, to orchestrate cell proliferation, differentiation, and survival. The MAPK signaling pathway is frequently dysregulated in human cancers, most commonly through mutation of members of the RAS family of genes. These mutations promote the GTP-bound state resulting in RAS activity leading in turn to activation of RAF, MEK, and ERK proteins. RAS mutations are found in multiple cancer types, including colorectal, lung, and pancreatic cancers.

RAF (Rapidly Accelerated Fibrosarcoma) is a serine-threonine protein kinase discovered as a retroviral oncogene. The RAF family of proteins (ARAF, BRAF, CRAF) signals just downstream of activated RAS. Activated GTP-bound RAS recruits cytosolic inactive RAF monomers to the plasma membrane where RAF binds to GTP-RAS thereby promoting homo- and heterodimerization of RAF. The dimerization of RAF facilitates conformational changes that lead to catalytically activated RAF. Activated RAF dimers phosphorylate and activate MEK1/2 (also known as mitogen-activated protein kinase) proteins, which subsequently phosphorylate and activate extracellular signal-regulated kinases (ERK1/2). ERKs phosphorylate a variety of substrates, including multiple transcription factors, thereby regulating several key cellular activities, including proliferation, metabolism, migration, and survival. The role of ERK1/2 in cancer is of special interest because activating mutations upstream of ERK1/2 in its signaling cascade are believed to be responsible for more than half of all cancers.

Dysregulated activation at any step in the MAPK pathway contributes to tumorigenesis. Activating BRAF mutations can be found in approximately 7% of cancers, with V600E accounting for greater than 90% of observed mutations in BRAF. The V600E mutation encodes a valine to glutamic acid substitution that exposes the active site of BRAF, enabling its constitutive activation as monomers or dimers independent of RAS. Inhibitors of active RAF, such as vemurafenib, dabrafenib, and encorafenib, have demonstrated dramatic activity in BRAF V600E metastatic melanoma with overall response rates (ORR) of 50-70%. The success of these inhibitors in V600E melanoma derives from the ability to bind to and inhibit the mutant monomeric form of RAF that is the oncogenic driver in cancer cells. However, in cancer cells that express wild-type BRAF, or in the normal cells of patients with V600E driven cancers, inhibitors such as vemurafenib paradoxically activate RAF signaling. The complexity of MAPK pathway signaling in the presence of monomeric RAF inhibitors is highlighted in patients whose BRAF V600E-dependent melanoma cells die while normal epidermal cells containing wild-type BRAF hyperproliferate. This paradoxical activation of RAF in wild-type cells is precipitated by the inhibitor's binding to one protomer of a RAF dimer. This leads to a conformational change that prevents inhibitor binding to the second protomer, and transactivation of the second RAF protomer of the dimer ensues. Inhibition at sequential nodes of the MAPK pathway with RAF- and MEK-directed combination therapy attenuates RAF dimer signaling in normal cells, thereby improving safety and clinical activity in metastatic BRAF V600 melanoma.

Single-agent RAF inhibitors or combination RAF/MEK inhibition in BRAF V600E colorectal cancer (CRC) demonstrate minimal activity; clinical benefit is limited compared to the activity seen in melanoma. Intrinsic and acquired resistance to RAF inhibitors and MEK inhibitors develop at multiple levels of the MAPK pathway. The complexities of signaling feedback and alternate pathways that circumvent BRAF inhibition are central to the challenge of targeting activated BRAF in CRC. Under physiologic conditions, activated MAPK signaling through mutant BRAF leads to ERK-dependent negative feedback on signals generated through activated RAS. Intrinsic resistance to RAF inhibition manifests because drugs such as vemurafenib or dabrafenib effectively inhibit BRAF V600E signaling through MEK to ERK; however, this in turn releases ERK-dependent negative feedback into RAS signaling. Therefore, upstream signals are able to activate RAS, leading to the induction of BRAF V600E and wild-type homo- and heterodimers. Because agents such as dabrafenib and vemurafenib inhibit V600E activated monomers in BRAF-dependent CRC cells, RAS-stimulated RAF dimer signaling is unopposed, leading to ERK reactivation to a greater degree than is seen in BRAF V600E melanoma, and thus limiting the effectiveness of therapy in CRC.

Under the pressure of RAF and MEK inhibition in BRAF V600E CRC, acquired resistance quickly develops. For instance, in an analysis of nine tumor samples from eight patients experiencing disease progression after MAPK inhibition, genetic alterations leading to MAPK reactivation were uncovered. These included activating mutations in KRAS or NRAS, amplification of wild-type (WT) NRAS or KRAS or mutant BRAF V600E, and an intragenic deletion in BRAF V600E. Acquired genetic alterations have also been reported, leading to reactivation of ERK signaling in the face of MAPK inhibitors. Acquired resistance may also arise through complementary signaling in the tumor microenvironment.

Though previous therapeutic approaches to BRAF-mutant CRC have focused on chemotherapy and/or targeted therapy, there is also a role for immunotherapy. During tumorigenesis, cancer cells exploit immune checkpoint pathways to avoid detection by the adaptive immune system. Monoclonal antibody (mAb) inhibitors of the Programmed Cell Death Protein-1 (PD-1) and Programmed Death-Ligand 1 (PD-L1) immunological checkpoints have demonstrated significant antitumor activity in patients with various solid tumors. PD-1 is a particularly important immunological target, with inhibitors such as pembrolizumab and nivolumab demonstrating single-agent activity in melanoma, non-small cell lung carcinoma (NSCLC), and other solid tumors.

CRC, however, is generally unresponsive to PD-1 blockade with the exception of tumors possessing microsatellite instability. There is, however, rationale for the use of small molecule inhibitors to modulate the immune response. The same therapies that inhibit genetic dependencies on the MAPK pathway in cancer cells inhibit signaling cascades in immune cells. For instance, preclinical studies demonstrated that MAPK pathway inhibitors, such as BRAF and MEK inhibitors, could improve lymphocyte homing and function by increasing tumor infiltrating lymphocytes in tumors.

Therefore, RAF and MEK inhibitors may modulate the immune response to tumors, and the combination of such agents with checkpoint blockade may increase the susceptibility of “immune cold” tumors such as CRC to PD-1 inhibition. Furthermore, approximately 20% of BRAF-mutant CRCs are characterized by genetic microsatellite instability (MSI-H: microsatellite instability-high). In MSI-H CRC, irrespective of BRAF genetic status, single-agent anti-PD-1 therapy has been associated with response rates of 30-50%.

Lung cancer is a common type of cancer that affects men and women around the globe. NSCLC is the most common type (roughly 85%) of lung cancer with approximately 70% of these patients presenting with advanced disease (Stage IIIB or Stage IV) at the time of diagnosis. About 30% of NSCLC tumors contain activating KRAS mutations, and these mutations are associated with resistance to EGFR tyrosine kinase inhibitors (TKIs). Activating KRAS mutations are also frequently found in melanoma, pancreatic cancer and ovarian cancer. BRAF mutations have been observed in up to 3% of NSCLC and have also been described as a resistance mechanism in EGFR mutation positive NSCLC.

CRC is a common disease with more than 1.8 million new cases estimated worldwide in 2018, along with >800,000 deaths (World Health Organization, Globocan 2018). Mutations in genes encoding components of the MAPK pathway are common, with RAS mutations occurring in approximately 50% of CRC. Activating mutations in the gene encoding BRAF V600E are present in approximately 10-15% of CRC patients, and mutated BRAF confers a poor prognosis. The V600E mutation occurs in approximately 90% of BRAF-mutant CRC, though others, for example, V600D or V600K mutations are also seen.

Effective treatment options for BRAF-mutant CRC are limited. Unlike melanoma, where single-agent BRAF inhibitors yielded responses rates of approximately 70% in the metastatic setting, single agent inhibition of metastatic BRAF-mutant CRC with vemurafenib was associated with an ORR of approximately 5%. Combination therapy with agents targeting the MAPK pathway have improved upon the effectiveness of BRAF inhibition, though outcomes are still poor. Dabrafenib combined with the MEK inhibitor trametinib was associated with an ORR of 12% and progression-free survival (PFS) of 3.5 months.

In CRC, stimulation of RAS through growth factor-mediated receptor tyrosine kinase activation supports the oncogenic milieu. Inhibitors of EGFR modestly improved upon the effectiveness of BRAF inhibition; BRAF inhibitors combined with EGFR inhibitors were associated with ORRs of 4-22% and PFS 3.2-4.2 months. Patients treated with dabrafenib +trametinib +panitumumab experienced an ORR of 21% and PFS of 4.2 months. In the phase III BEACON trial, patients were randomized to one of three arms in the 2nd-line of treatment or higher: encorafenib/binimetinib/cetuximab, encorafenib/cetuximab, versus irinotecan/cetuximab or FOLFIRI/cetuximab (control). Patients receiving triplet therapy achieved an ORR of 26%, PFS of 4.3 months, and overall survival (OS) of 9 months. Encorafenib plus cetuximab was associated with an ORR of 20% and PFS of 4.2 months, and OS of 8.4 months. Both regimens achieved statistically significant improvements over irinotecan or FOLFIRI/cetuximab, which was associated with an ORR of 2%, a PFS of 1.5 months, and OS of 5.4 months. The improved outcomes demonstrated by combined inhibition of RAF, MEK, and EGFR signaling support the concept that inhibition of multiple nodes within the MAPK pathway is required for the treatment of BRAF V600E CRC.

Dabrafenib (Tafinlar®) is an orally bioavailable, potent and selective inhibitor of RAF kinases, whose mechanism of action of is consistent with competitive inhibition of adenosine triphosphate (ATP) binding. The ability of dabrafenib to inhibit some mutated forms of BRAF kinases is concentration dependent, with in vitro IC50 values of 0.65, 0.5, and 1.84 nM for BRAF V600E, BRAF V600K, and BRAF V600D enzymes, respectively. Inhibition of wild-type BRAF and CRAF kinases requires higher concentrations, with IC50 values of 3.2 and 5.0 nM, respectively. Other kinases such as SIK1, NEK11, and LIMK1 may also be inhibited at higher concentrations. Dabrafenib inhibits cell growth of various BRAF V600 mutation-positive tumors in vitro and in vivo.

Dabrafenib was first approved by the FDA in 2013 as a single-agent oral treatment for unresectable or metastatic melanoma in adult patients with the BRAF V600 mutation and is approved in various other countries for the same indication. Dabrafenib in combination with trametinib is also approved in multiple countries for the following indications (approved indications vary by country): treatment of patients with unresectable or metastatic melanoma with a BRAFV600 mutation; the adjuvant treatment of patients with Stage III melanoma with a BRAFV600 mutation, following complete resection; treatment of patients with advanced non-small cell lung cancer (NSCLC) with a BRAFV600 mutation; and treatment of patients with locally advanced or metastatic anaplastic thyroid cancer (ATC) with a BRAFV600E mutation.

The recommended dose of dabrafenib is 150 mg BID (corresponding to a total daily dose of 300 mg).

Compound A is a potent, selective and orally bioavailable ATP-competitive ERK1/2 kinase inhibitor that exhibits physical chemical properties enabling combinations with RAF and MEK inhibitors, or other targeted therapeutic agents. Compound A effectively inhibits pERK signaling and has demonstrated tumor growth inhibition in multiple MAPK-activated cancer cells and xenograft models. Importantly, compound A demonstrated broad efficacy targeting multiple known mechanisms of resistance to BRAF and MEK inhibitors, including RAS mutations, BRAF splice variants and MEKI/2 mutations, as shown in engineered cell line models. Compound A has been dosed in patients between 45 mg and 450 mg QD.

Clinical studies in BRAF V600E CRC have demonstrated that the activity of RAF inhibitors alone or in combination with MEK f EGFR inhibitors is limited by insufficient MAPK pathway suppression, and that in patients, mechanisms of resistance quickly arise even in the setting of initial clinical benefit. Acquired resistance mechanisms leading to MAPK pathway reactivation in patient tumors primarily involve activating genetic alterations in RAS, BRAF or MEK. This highlights the reliance of BRAF V600E CRC on MAPK signaling, and suggests that inhibition of ERK, the most downstream point of the signaling pathway, may circumvent resistance occurring at upstream nodes.

Preclinical models of RAS, RAF, or MEK resistance mutations engineered into a BRAF V600E cell line supported this concept. While the parental BRAF V600E cell line was sensitive to combinations of BRAF, MEK, EGFR, and/or ERK inhibitors, the introduction of KRAS, NRAS, MEK1, or MEK2 resistance mutations resulted in decreased sensitivity of engineered BRAF V600E cells to all inhibitor combinations, except for those containing an ERK inhibitor. Furthermore, the outgrowth of pre-existing, low-frequency pooled resistant clones in mouse xenografts was suppressed more effectively by treatment with drug combinations containing BRAF and ERK inhibitors, as compared to BRAF and MEK inhibitors.

The combination of Dabrafenib+Compound A was tested in vivo in the BRAF mutant human cell line xenograft HT29. Mice treated with Dabrafenib+Compound A achieved similar anti-tumor response as compared to Dabrafenib+Trametinib at clinically relevant doses (36% T/C vs 28% T/C, respectively). Single agent treatment led to progressive disease, whereby compound A achieved 54% T/C, Dabrafenib achieved 59% T/C, and Trametinib achieved 48% T/C. All regimens were tolerated as judged by lack of significant body weight loss. These data suggest that the combination of Dabrafenib+Compound A may achieve similar anti-tumor activity to Dabrafenib+Trametinib in patients with BRAF mutant colorectal cancer, and provides rationale for its use in the clinic.

The improved outcomes demonstrated by combined inhibition of RAF, MEK, and EGFR signaling support the concept that inhibition of multiple nodes within the MAPK pathway is required for the treatment of BRAF V600 CRC.

Nonetheless, intrinsic and acquired resistance to therapy remain important challenges, and clinical outcomes are still poor. There is a role for combination therapies that provide more robust suppression of MAPK signaling and address the complexity of mechanisms of resistance both within and beyond the MAPK pathway. Given the adaptive complexity of signal transduction that characterizes BRAF-mutant CRC, inhibition of proteins beyond RAF and ERK is required. As an illustration, one study of 218 BRAF-V600E mutated CRC tumors identified distinct subsets of tumors characterized by high KRAS/mTOR/AKT/4EBP1/EMT activation, while cell-cycle dysregulation characterized the other subset.

Despite the advances demonstrated by targeted therapy combinations, such as those studied in the BEACON trial (Kopetz et al. 2019), the ability to shut down the BRAF V600 oncogenic drive in cancer cells is limited by 1.) the inability to fully suppress RAF activity due the adaptive ability of RAF kinases to signal through ineffectively inhibited dimers, and 2.) ongoing ERK activation stimulated not only by adaptive mechanisms within the MAPK pathway, but also through parallel signaling pathways. Dabrafenib, vemurafenib, and encorafenib effectively suppress RAF activity in BRAF-mutant cancer cells where monomeric V600E is an oncogenic driver. However, these drugs may also lead to the paradoxical activation of ERK through several mechanisms.

Combined inhibition of RAF and MEK improves upon pathway suppression; however, the persistence of ERK signaling underlies the limitations of this therapeutic approach. Blockade of ERK, the ultimate signal of the MAPK pathway, may circumvent adaptive upstream signals and provide for improved efficacy and resilience to acquired resistance.

SHP2 is a phosphatase that binds activated RTKs and transduces their signaling downstream to the RAS/MAPK and PI3K/AKT pathways. Inhibition of SHP2 therefore inhibits RTK-mediated signaling. SHP2 is also known to regulate PI3K, Fak, RhoA, Ca2+ oscillations, Ca2+/Calcineurin and NFAT signaling, and SHP2 also acts downstream of cytokine signaling in the regulation of Jak/Stat signaling. In addition, SHP2 signals downstream of the immune checkpoint molecule PD-1, B- and T-lymphocyte attenuator (BTLA), and indoleamine 2,3-dioxygenase (IDO). Thus, SHP2 has RAS/MAPK-independent functions in tumorigenesis by regulating neoplastic migration, invasion, metastasis, or anti-tumor immune response.

Clinical studies adding anti-EGFR antibodies to RAF and MEK inhibition have demonstrated modestly improved outcomes in BRAF V600 CRC. Preclinical studies, however, suggest that other RTK pathways may contribute to signal activation in the setting of BRAF V600 CRC. SHP2 plays a central role in mediating signals emanating from not only EGFR, but also from other RTKs, and therefore has the potential to expand upon the activity of drugs such as cetuximab and panitumumab when combined with inhibitors of the MAPK pathway. Therefore, SHP2 inhibition can provide more effective initial MAPK pathway suppression and also better address mechanisms of MAPK pathway reactivation. The triple combination of dabrafenib+Compound A+Compound B can inhibit the MAPK pathway in BRAF V600 colorectal cancer by leveraging the potential to uniquely target mechanisms of intrinsic and acquired resistance in BRAF V600-driven cancer cells.

### Pharmaceutical Compositions

In another aspect, the present invention provides pharmaceutically acceptable compositions which comprise a therapeutically-effective amount of dabrafenib, compound A and compound B, formulated together with one or more pharmaceutically acceptable carriers (additives) and/or diluents. As described in detail below, the pharmaceutical compositions of the present invention may be specially formulated for administration in solid or liquid form, including those adapted for oral administration, for example, drenches (aqueous or non-aqueous solutions or suspensions), tablets, e.g., those targeted for buccal, sublingual, and systemic absorption, boluses, powders, granules, pastes for application to the tongue.

The phrase “pharmaceutically-acceptable carrier” as used herein means a pharmaceutically-acceptable material, composition or vehicle, such as a liquid or solid filler, diluent, excipient, manufacturing aid (e.g., lubricant, talc magnesium, calcium or zinc stearate, or steric acid), or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; and (22) other non-toxic compatible substances employed in pharmaceutical formulations.

As set out above, certain embodiments of the present compounds may contain a basic functional group, such as amino or alkylamino, and are, thus, capable of forming pharmaceutically-acceptable salts with pharmaceutically-acceptable acids. The term “pharmaceutically-acceptable salts” in this respect, refers to the relatively non-toxic, inorganic and organic acid addition salts of compounds of the present invention. These salts can be prepared in situ in the administration vehicle or the dosage form manufacturing process, or by separately reacting a purified compound of the invention in its free base form with a suitable organic or inorganic acid, and isolating the salt thus formed during subsequent purification. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, napthylate, mesylate, glucoheptonate, lactobionate, and laurylsulphonate salts and the like.

The pharmaceutically acceptable salts of the subject compounds include the conventional nontoxic salts or quaternary ammonium salts of the compounds, e.g., from non-toxic organic or inorganic acids. For example, such conventional nontoxic salts include those derived from inorganic acids such as hydrochloride, hydrobromic, sulfuric, sulfamic, phosphoric, nitric, and the like; and the salts prepared from organic acids such as acetic, propionic, succinic, glycolic, stearic, lactic, malic, tartaric, citric, ascorbic, palmitic, maleic, hydroxymaleic, phenylacetic, glutamic, benzoic, salicyclic, sulfanilic, 2-acetoxybenzoic, fumaric, toluenesulfonic, methanesulfonic, ethane disulfonic, oxalic, isothionic, and the like.

In other cases, the compounds of the present invention may contain one or more acidic functional groups and, thus, are capable of forming pharmaceutically-acceptable salts with pharmaceutically-acceptable bases. The term “pharmaceutically-acceptable salts” in these instances refers to the relatively non-toxic, inorganic and organic base addition salts of compounds of the present invention. These salts can likewise be prepared in situ in the administration vehicle or the dosage form manufacturing process, or by separately reacting the purified compound in its free acid form with a suitable base, such as the hydroxide, carbonate or bicarbonate of a pharmaceutically-acceptable metal cation, with ammonia, or with a pharmaceutically-acceptable organic primary, secondary or tertiary amine. Representative alkali or alkaline earth salts include the lithium, sodium, potassium, calcium, magnesium, and aluminum salts and the like. Representative organic amines useful for the formation of base addition salts include ethylamine, diethylamine, ethylenediamine, ethanolamine, diethanolamine, piperazine and the like.

A particularly preferred salt of dabrafenib is the mesylate salt thereof. A particularly preferred solvate of compound A is the hydrochloride salt thereof. A particularly preferred solvate of compound B is the succinate salt thereof.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically-acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Formulations of the present invention include those suitable for oral, nasal, topical (including buccal and sublingual), rectal, vaginal and/or parenteral administration. The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient which can be combined with a carrier material to produce a single dosage form will generally be that amount of the compound which produces a therapeutic effect. Generally, out of one hundred per cent, this amount will range from about 0.1 percent to about ninety-nine percent of active ingredient, preferably from about 5 percent to about 70 percent, most preferably from about 10 percent to about 30 percent.

In certain embodiments, a formulation of the present invention comprises an excipient selected from the group consisting of cyclodextrins, celluloses, liposomes, micelle forming agents, e.g., bile acids, and polymeric carriers, e.g., polyesters and polyanhydrides; and a compound of the present invention. In certain embodiments, an aforementioned formulation renders orally bioavailable a compound of the present invention.

Methods of preparing these formulations or compositions include the step of bringing into association a compound of the present invention with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules, cachets, pills, tablets, lozenges (using a flavored basis, usually sucrose and acacia or tragacanth), powders, granules, or as a solution, suspension or solid dispersion in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. A compound of the present invention may also be administered as a bolus, electuary or paste.

In solid dosage forms of the invention for oral administration (capsules, tablets, pills, dragees, powders, granules, trouches and the like), the active ingredient is mixed with one or more pharmaceutically-acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds and surfactants, such as poloxamer and sodium lauryl sulfate; (7) wetting agents, such as, for example, cetyl alcohol, glycerol monostearate, and non-ionic surfactants; (8) absorbents, such as kaolin and bentonite clay; (9) lubricants, such as talc, calcium stearate, magnesium stearate, solid polyethylene glycols, sodium lauryl sulfate, zinc stearate, sodium stearate, stearic acid, and mixtures thereof; (10) coloring agents; and (11) controlled release agents such as crospovidone or ethyl cellulose. In the case of capsules, tablets and pills, the pharmaceutical compositions may also comprise buffering agents. Solid compositions of a similar type may also be employed as fillers in soft and hard-shelled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions of the present invention, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be formulated for rapid release, e.g., freeze-dried. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions which can be used include polymeric substances and waxes. The active ingredient can also be in micro-encapsulated form, if appropriate, with one or more of the above-described excipients.

Liquid dosage forms for oral administration of the compounds of the invention include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Examples of suitable aqueous and nonaqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms upon the subject compounds may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions.

When the compounds of the present invention are administered as pharmaceuticals, to humans and animals, they can be given per se or as a pharmaceutical composition containing, for example, 0.1 to 99% (more preferably, 10 to 30%) of active ingredient in combination with a pharmaceutically acceptable carrier.

The compounds of the present invention and/or the pharmaceutical compositions of the present invention, are formulated into pharmaceutically-acceptable dosage forms by conventional methods known to those of skill in the art.

Actual dosage levels of the active ingredients in the pharmaceutical compositions of this invention may be varied so as to obtain an amount of the active ingredient which is effective to achieve the desired therapeutic response for a particular patient, composition, and mode of administration, without being toxic to the patient.

The selected dosage level will depend upon a variety of factors including the activity of the particular compound of the present invention employed, or the ester, salt or amide thereof, the route of administration, the time of administration, the rate of excretion or metabolism of the particular compound being employed, the rate and extent of absorption, the duration of the treatment, other drugs, compounds and/or materials used in combination with the particular compound employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved.

In general, a suitable daily dose of the combination of the invention will be that amount of each compound which is the lowest dose effective to produce a therapeutic effect. Such an effective dose will generally depend upon the factors described above.

In another aspect, the present invention provides pharmaceutically acceptable compositions which comprise a therapeutically-effective amount of one or more of the subject compounds, as described above, formulated together with one or more pharmaceutically acceptable carriers (additives) and/or diluents.

### EXAMPLES

### Example 1

### Dabrafenib, Compound A and Compound B

Dabrafenib is synthesized according to example 58a of WO2009/137391. Compound A is synthesized according to example 184 of WO2015/066188. Compound B is synthesized according to example 69 of WO2015/107495. WO2009/137391, WO2015/066188 and WO2015/107495, are herein incorporated by reference in their entirety.

The utility of a combination of Dabrafenib, Compound A and Compound B described herein can be evidenced by testing in the following examples.

### Example 2

Combination Activity of MAPK Pathway Inhibitors in BRAF-Mutant CRC Cell Lines

Dabrafenib (DRB436): selective inhibitor of mutated BRAF at V600 capable of inhibiting BRAF(V600E), BRAF(V600K) and BRAF(V600G) mutations. Compound A: selective ATP-competitive ERK1 and ERK2 kinase inhibitor. Compound B: selective allosteric inhibitor of SHP2. The compounds were dissolved in 100% DMSO and stored at -20° C. as 10 mM stock solutions.

In this study, we used six BRAF-mutant colorectal cancer cell lines. We acquired all the cell lines from ATCC and cultured them at 37° C. 5% CO₂ in the recommended media conditions: Colo205, LIM2405, SNUC5 and SW1417: RPMI 1640 (Amimed, #1-41F01-I) supplemented with 1% L-glutamine, 10 mM HEPES (Amimed, #5-31F00-H), 1% Na-pyruvate (Amimed, #5-60F00-H), 10% FCS. MDST8: DMEM high glucose (Amimed, #1-26F01-I) supplemented with 1% L-glutamine, 10% FCS. RKO: EMEM (Amimed, #1-31S01-1) supplemented with 1% L-glutamine, 10% FCS.

Cell lines were dispensed into tissue culture treated 384-well plates (Greiner #781098) in a final volume of 25 μl per well and a concentration of 500 cells per well. Cells were allowed to adhere and begin growth for twenty-four hours. Compound dilutions or DMSO were added using a HP D300 digital dispenser. After seventy-two hours the medium was refreshed by supplementing 25 μl per well of culture medium containing the corresponding compound dilutions or DMSO.

Seven days after treatment initiation, cell growth was determined using CellTiter-Glo® (Promega, #G7573), which measures the amount of ATP in the well. Plates were equilibrated to room temperature for approximately thirty minutes and one volume of CellTiter-Glo® Reagent equal to the volume of cell culture medium was added. Cell lysis was induced for two minutes on an orbital shaker, the plates were incubated at room temperature for ten minutes, and luminescence was recorded.

To summarize the data of this study clearly, the percentage of growth inhibition versus DMSO (% GI) at single compound concentrations were reported. These concentrations reflect the clinically achievable concentrations in patients: 30 nM for dabrafenib and 300 nM for Compound A. Concentration of compound B was 1.1 μM; a concentration of compound that is active and selective for SHP2 in cell assays. Raw data values were normalized to day 0, the time of treatment initiation, so that the % GI could be calculated. Formula used for % GI was [(compound-treated cells at day 7−cells at day 0)/(DMSO-treated cells at day 7−cells at day 0)]×100%; where day 0=cells prior to treatment. Reported is the mean and standard deviation for between three and sixteen experiments. Between group comparisons were carried out using a one-way ANOVA followed by a Tukey's multiple comparisons test. For all statistical evaluations the level of significance was set at p<0.05.

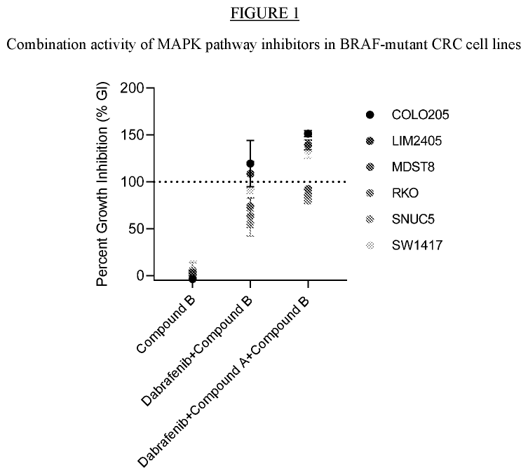

The ability of Compound B to control the in vitro growth and survival of BRAFV600E CRC cell lines in the presence of dabrafenib and Compound A was analyzed in six BRAFV600E CRC cell lines. While Compound B monotherapy had no effect on cell growth inhibition, when combined with dabrafenib+Compound A it significantly enhanced cell growth inhibition and/or cell kill in all cell lines (See FIG. 1). In FIG. 1, six BRAF-mutated CRC cell lines were treated with the either Compound B alone, dabrafenib+compound A doublet or dabrefenib+Compound A+Compound B triplet. The graph shows the percentage of growth inhibition (% GI) achieved after seven treatment days with respect to DMSO-treated cells. The % GI values are average values of independent experiments and the vertical error bars indicate the standard deviation. The horizontal dotted line indicates 100% GI (cell stasis). Values extending beyond 100% GI indicate cell kill.

The addition of Compound B to SNUC5 (p=0.0061), RKO (P=0.0039) and MDST8 (P=0.0013) cells led to a significantly greater inhibition of growth with dabrafenib+Compound A compared to the doublet of dabrefnib+Compound A. Dabrafenib+Compound A led to growth stasis of SW1417 cells, and the addition of Compound B to dabrafenib+Compound A caused cell culture regression. The addition of Compound B to LIM2405 (p=0.0003) and COLO205 (p=0.0369) cells resulted in significantly enhanced cell kill compared to dabrafenib+Compound A doublet.

In summary, for the six BRAF V600E mutant CRC cell lines growing in vitro, the addition of Compound B to dabrafenib +Compound A resulted in enhanced benefit in all six BRAF-mutant CRC cell lines and led to significantly better control of cell growth and/or greater cell killing. These data show that better suppression of RTK-mediated feedback activation via SHP2 inhibition can better control BRAF-mutant CRC growth and survival. These results show that triple combinations of MAPK pathway inhibition is required to completely and durably block MAPK signaling, and therefore cancer cell growth and survival, in clinical disease.

It is understood that the Examples and embodiments described herein are for illustrative purposes only and that various modifications or changes in light thereof will be suggested to persons skilled in the art and are to be included within the spirit and purview of this application and scope of the appended claims.

What is claimed is:

1. A pharmaceutical combination comprising: N-(3-(5-(2-aminopyrimidin-4-yl)-2-(tert-butyl)thiazol-4-yl)-2-fluorophenyl)-2,6-difluorobenzenesulfonamide (dabrafenib), or a pharmaceutically acceptable salt thereof; 4-(3-amino-6-((1 S,3 S,4S)-3-fluoro-4-hydroxycyclohexyl)pyrazin-2-yl)-N-((S)-1-(3-bromo-5-fluorophenyl)-2-(methylamino)ethyl)-2-fluorobenzamide (compound A), or a pharmaceutically acceptable salt thereof; and (3S,4S)-8-(6-amino-5-((2-amino-3-chloropyridin-4-yl)thio)pyrazin-2-yl)-3-methyl-2-oxa-8-azaspiro[4.5]decan-4-amine (compound B), or a pharmaceutically acceptable salt thereof.

2. The combination of claim 1, wherein N-(3-(5-(2-aminopyrimidin-4-yl)-2-(tert-butyl)thiazol-4-yl)-2-fluorophenyl)-2,6-difluorobenzenesulfonamide (dabrafenib), or a pharmaceutically acceptable salt thereof, 4-(3-amino-6-((1S,3S,4S)-3-fluoro-4-hydroxycyclohexyl)pyrazin-2-yl)-N-((S)-1-(3-bromo-5-fluorophenyl)-2-(methylamino)ethyl)-2-fluorobenzamide (compound A), or a pharmaceutically acceptable salt thereof, and (3S,4S)-8-(6-amino-5-((2-amino-3-chloropyridin-4-yl)thio)pyrazin-2-yl)-3-methyl-2-oxa-8-azaspiro[4.5]decan-4-amine (compound B), or a pharmaceutically acceptable salt thereof, are administered separately, simultaneously or sequentially, in any order.

3. The pharmaceutical combination according to claim 1 or 2, which is for oral administration.

4. The pharmaceutical combination according to any one of claims 1 to 3, wherein N-(3-(5-(2-aminopyrimidin-4-yl)-2-(tert-butyl)thiazol-4-yl)-2-fluorophenyl)-2,6-difluorobenzenesulfonamide (dabrafenib) is in an oral dosage form.

5. The pharmaceutical combination according to any one of claims 1 to 4, wherein 4-(3-amino-6-((1 S,3 S,4S)-3-fluoro-4-hydroxycyclohexyl)pyrazin-2-yl)-N-((S)-1-(3-bromo-5-fluorophenyl)-2-(methylamino)ethyl)-2-fluorobenzamide (compound A) is in an oral dosage form.

6. The pharmaceutical combination according to any one of claims 1 to 4, wherein (3S,4S)-8-(6-amino-5-((2-amino-3-chloropyridin-4-yl)thio)pyrazin-2-yl)-3-methyl-2-oxa-8-azaspiro[4.5]decan-4-amine (compound B) is in an oral dosage form.

7. A pharmaceutical composition or a commercial package comprising the pharmaceutical combination according to any one of the preceding claims and at least one pharmaceutically acceptable carrier.

8. A pharmaceutical combination according to any one of claims 1 to 6 or the pharmaceutical composition or the commercial package according to claim 7 for use in the treatment of cancer.

9. The pharmaceutical combination or the pharmaceutical composition or the commercial package for use according to claim 8, wherein the cancer is selected from breast cancer, cholangiocarcinoma, colorectal cancer (CRC), melanoma, non-small cell lung cancer, ovarian cancer and thyroid cancer.

10. The pharmaceutical combination or the pharmacutical composition or the commercial package for use according to claim 8, wherein the cancer is advanced or metastatic colorectal cancer.

11. The pharmaceutical combination of claim 10 wherein the cancer is BRAF gain of function CRC or BRAF V600E, V600D or V600K CRC.

12. Use of the pharmaceutical combination according to any one of claims 1 to 6 or the pharmaceutical composition or commercial package according to claim 7 for the manufacture of a medicament for the treatment of cancer.

13. The use of the pharmaceutical combination or the pharmaceutical composition according to claim 12 wherein the cancer is selected from breast cancer, cholangiocarcinoma, colorectal cancer, melanoma, non-small cell lung cancer, ovarian cancer and thyroid cancer, optionally wherein the cancer is advanced or metastatic colorectal cancer, optionally wherein the cancer is BRAF gain of function CRC or BRAF V600E, V600D or V600K CRC.

14. A method of treating a cancer selected from breast cancer, cholangiocarcinoma, colorectal cancer, melanoma, non-small cell lung cancer, ovarian cancer and thyroid cancer comprising administrating to a patient in need thereof a pharmaceutical combination or commercial package according to any one of claims 1 to 6 or the pharmaceutical composition according to claim 7.

15. The method of claim 14 wherein the colorectal cancer is advanced or metastatic colorectal cancer.

16. The method of claim 15 wherein the colorectal cancer is BRAF gain of function CRC or BRAF V600E, V600D or V600K CRC.

17. The method of claim 14, wherein N-(3-(5-(2-aminopyrimidin-4-yl)-2-(tert-butyl)thiazol-4-yl)-2-fluorophenyl)-2,6-difluorobenzenesulfonamide (dabrafenib) is administered orally at a dose of about from about 1 to about 150 mg per day.

18. The method of claim 14, wherein 4-(3-amino-6-((1S,3S,4S)-3-fluoro-4-hydroxycyclohexyl)pyrazin-2-yl)-N-((S)-1-(3-bromo-5-fluorophenyl)-2-(methylamino)ethyl)-2-fluorobenzamide (compound A) is administered orally at a dose of from about 50 to about 200 mg per day.

19. The method of claim 14, wherein (3S,4S)-8-(6-amino-5-((2-amino-3-chloropyridin-4-yl)thio)pyrazin-2-yl)-3-methyl-2-oxa-8-azaspiro[4.5]decan-4-amine (compound B) is adminstered orally at a dose of from about 1.5 mg per day, or 3 mg per day, or 6 mg per day, or 10 mg per day, or 20 mg per day, or 30 mg per day, or 40 mg per day, or 50 mg per day, or 60 mg per day to about 70 mg per day.

20. The method of claim 19 wherein the dose per day is on a 21 day cycle of 2 weeks on drug followed by 1 week off drug.
